(19) United States
(12) Patent Application Publication
(10) Pub. No.: US 2021/0322657 A1
(43) Pub. Date: Oct. 21, 2021

(54) CONTAINER AND DIALYSIS MACHINE COMPRISING A CONTAINER

(71) Applicant: FRESENIUS MEDICAL CARE DEUTSCHLAND GMBH, Bad Homburg (DE)

(72) Inventor: Jenspeter RAU, Munich (DE)

(21) Appl. No.: 17/231,025

(22) Filed: Apr. 15, 2021

(30) Foreign Application Priority Data
Apr. 20, 2020 (DE) 10 2020 110 669.6

(51) Int. Cl. A61M 1/16; A61M 39/10

(52) U.S. Cl. CPC A61M 1/167; A61M 39/10

(57) ABSTRACT

The present invention relates to a container for receiving a dry concentrate for producing a dialysis solution, comprising a container opening and a connector arranged on the container opening, the container comprising a side wall and the container opening being arranged in this side wall, and the connector comprising fixing means for fixing the container to a dialysis machine, which comprises a first retaining element having a container inlet and a second retaining element having a container outlet, a connection element being provided on which the first and the second retaining element are arranged and which is provided with lines that are fluidically connected to the container inlet and outlet, and a housing element of the connector being provided which is fluidically connected both to said lines and to the container opening and extends from the connection element to the container opening, the housing element comprising a curved or angled portion, wherein the connection element does not comprise a widened central portion in a plan view of the container.

The present application claims priority based on German Patent Application No. 10 2020 110 669.6, filed Apr. 20, 2020, the contents of which are incorporated herein by reference.

The present invention relates to a container for receiving a dry concentrate for producing a dialysis solution, comprising a container opening and a connector arranged on the container opening, the container comprising a side wall and the container opening being arranged in this side wall, and the connector comprising fixing means for fixing the container to a dialysis machine, which comprises a first retaining element having a container inlet and a second retaining element having a container outlet, a connection element being provided on which the first and the second retaining element are arranged and which is provided with lines that are fluidically connected to the container inlet and outlet, and a housing element of the connector being provided which is fluidically connected both to said lines and to the container opening and extends from the connection element to the container opening, the housing element comprising a curved or angled portion.

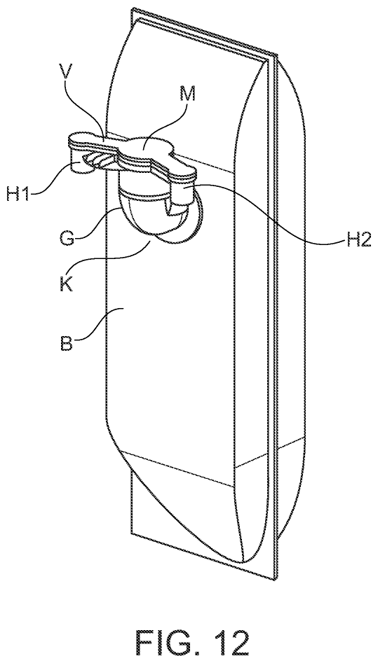

A container of this kind is known from CN 110613868 A. FIG. 12 shows the container B disclosed in this document, in the side wall of which a connector K is arranged which comprises retaining elements H1 and H2. The connection element V extends between said elements. The housing element G, which extends from the connection element V to the container opening, extends downwards starting from the connection element V. As can be seen from FIG. 12, the central region M is thickened compared with the other regions of the connection element, i.e., in a plan view, has a greater width than the adjacent or other regions of the connection element V.

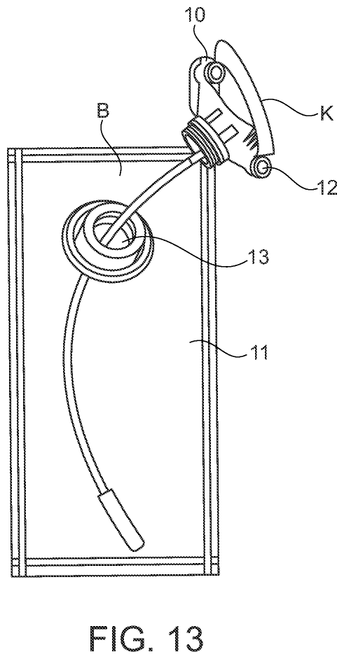

FIG. 13 shows another container known from the prior art. FIG. 13 shows the liquid inlet and outlet via the retaining elements 10, 12 of the connector K. Said elements are vertical relative to the bag inlet, i.e. the container opening 13, the inlet and outlet being positioned through the retaining elements 10, 12 in a plane below the inlet 13 to the bag. The inlet to the bag 11, i.e. the bag opening 13, is positioned on a side surface of the bag, as is clear from FIG. 13.

The problem addressed by the invention is to provide a material-saving container of which the connector requires little space.

This problem is solved by a container having the features of claim 1. Accordingly, it is provided that the connection element does not comprise a widened central portion in a plan view of the container. The central portion of the connection element is therefore not designed to be wider than regions adjacent to the central portion and/or than other regions of the connection element, such as projections, which extend from the central portion to the respective retaining elements.

The liquid to be supplied to the container or another fluid, e.g. gas or RO water, enters the first retaining element from the machine-side inlet, enters the connection element therefrom, and enters the interior of the container therefrom. The dialysis solution enters the connection element from the container, and from there enters the second retaining element, and enters the machine-side outlet therefrom.

When the bag is mounted, the retaining elements thereof are fluidically coupled to the dialysis-machine-side inlet and outlet.

The term “upwards” or “at the top” relates to the usual usage position of the container that it assumes when it is used, i.e. when it is connected to a dialysis machine.

The term “dialysis solution” covers both the ready-to-use dialysis solution and a part thereof, such as a concentrate.

The fluid(s) may be gas and/or liquid.

According to the invention, it is provided that the container comprises a side wall and that the container opening is arranged in this side wall. The container opening is therefore not positioned at the top of the container, but on the side of said container.

The connection element may comprise a first and a second projection, the first projection being connected to the first retaining element and the second projection being connected to the second retaining element.

It is preferably provided here that a first edge or upper edge of the first projection and a first upper edge of the second projection transition into the central portion without widening and/or in that a second edge or lower edge of the first projection and a second lower edge of the second projection transition into the central portion without widening.

In an embodiment, the central portion and said projections have the same width in a plan view of the container.

The connection element may be designed to be angled or curved in a plan view of the container. The projections and the central portion may be arranged in a V shape or may together form a V.

It is also conceivable and covered by the invention that the projections and the central portion are arranged on a line, i.e. that the central portion and the projections are arranged linearly relative to one another.

In another embodiment, the connection element comprises a planar upper face which is arranged on the side of the connection element opposite the side of the connection element on which the housing element is arranged.

Furthermore, it is also provided that the retaining elements are arranged so as to be offset upwards relative to the container opening. This has the advantage of improving the accessibility of elements arranged behind the bag or container during use. The upper face of the connector may be arranged to be higher than the upper edge of the bag or container.

The housing element may have a consistent or increasing diameter and/or cross section starting from the connection element towards the container opening. It may be circle-segment-shaped, preferably quarter-circle-shaped, or may comprise a portion of this kind.

In another embodiment of the invention, the retaining elements may have a first orientation, and the surface normal of the container opening has a second orientation, said two above-mentioned orientations not extending in parallel, but rather at an angle, and preferably at a right angle, to one another. As a result, the container may be arranged in parallel with the retaining elements and/or with a dialysis machine when the container is connected.

The upper face of the connector may be closed by a film or a cover. The film and cover may be firmly welded on.

The connector may comprise a separating means which is arranged to separate the liquid that enters the vessel through the container inlet from the liquid discharged from the vessel through the container outlet, such that the probability of a short-circuit flow occurring is minimized.

The ends of the lines of the connection element may be arranged one behind the other or beside one another in a front view of the container.

The ends of the lines of the connection element facing the interior of the container are therefore preferably arranged one behind the other or beside one another in a front view of the container.

The container may comprise fixed and/or flexible container walls. The container is preferably designed as a bag. The use of a cartridge is also conceivable and is covered by the invention.

The present invention relates both to the container comprising a connector per se (i.e. when the bag is empty) and to a container in which solid or liquid dialysis concentrate or a dialysis solution is located.

The connector may be designed in one piece, i.e. the retaining elements, the connection element and the housing element may be one-piece components of the connector. A multiple-piece arrangement is also covered by the invention.

The present invention further relates to a dialysis machine comprising a first connection for supplying the container with liquid and comprising a second connection for discharging liquid from the vessel, characterized in that the first and the second retaining element of a container according to any of claims 1 to 16 are arranged at the first and the second connection, respectively.

It should be noted at this point that the terms “a”, “an” and “one” do not necessarily refer to exactly one of the elements, even though this constitutes a possible configuration, but instead they can also denote a plurality of said elements. Likewise, the use of the plural also includes the presence of the element in question in the singular and, vice versa, the singular also includes a plurality of the elements in question.

Other details and advantages of the invention are explained in greater detail with reference to an embodiment shown in the drawings, in which:

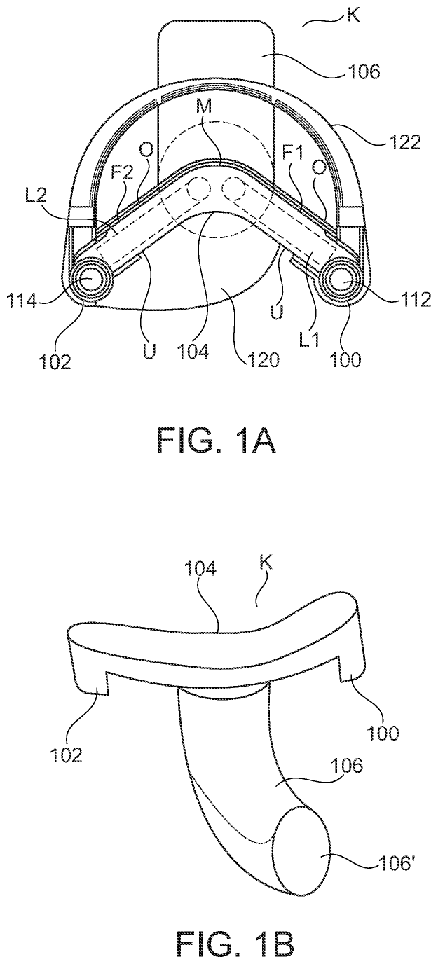

FIGS. 1A-B, 2A-B, and 3A-B are cross-sectional views and perspective views of a connector according to the invention in different embodiments,

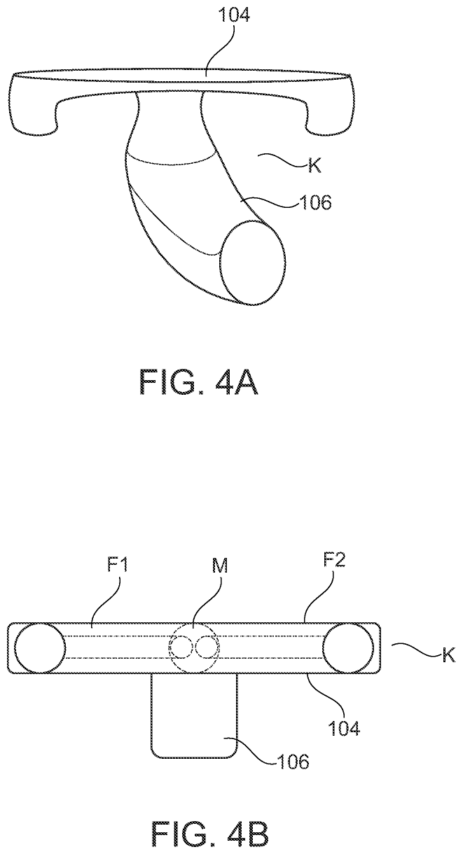

FIGS. 4A-B, 5A-B, and 6A-B are further cross-sectional views and perspective views of a connector according to the invention in different embodiments,

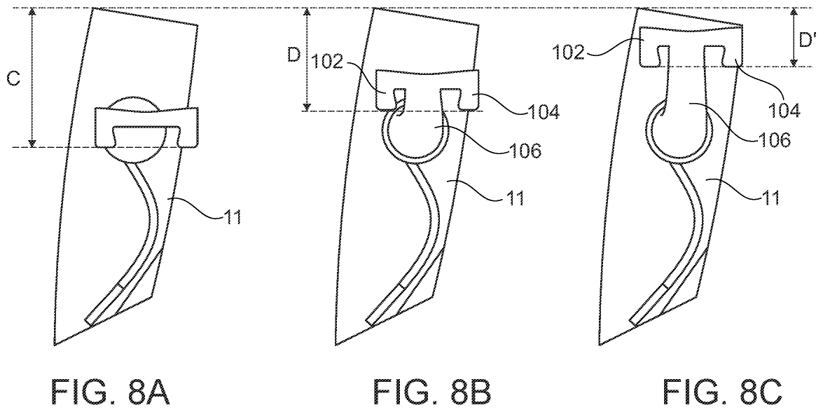

FIG. 7 is a perspective view of bag with a detailed view of the connector according to the invention,

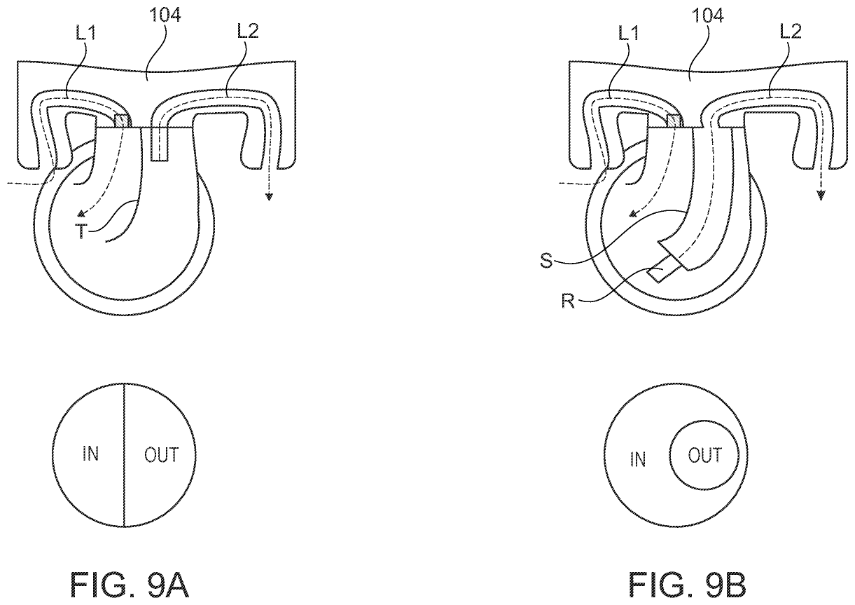

FIGS. 8A-C show perspective views of a bag according to the prior art and according to the invention,

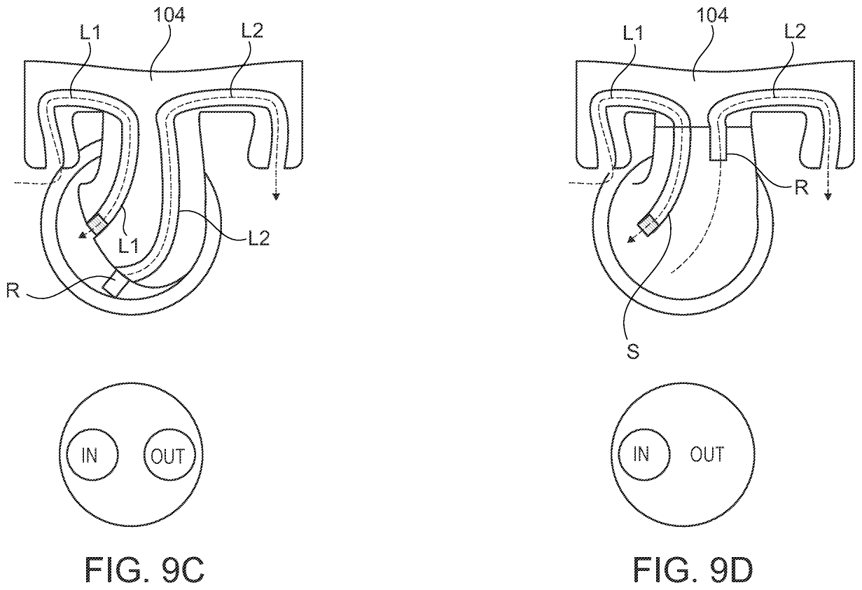

FIGS. 9A-E show longitudinal and cross-sectional views of different designs of connectors,

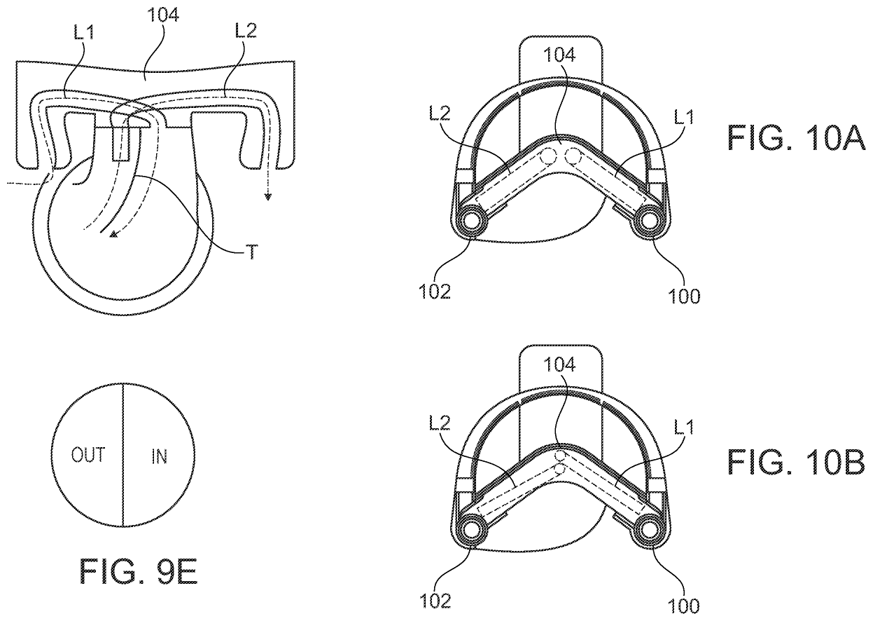

FIGS. 10A-B show cross-sectional views through bags according to the invention,

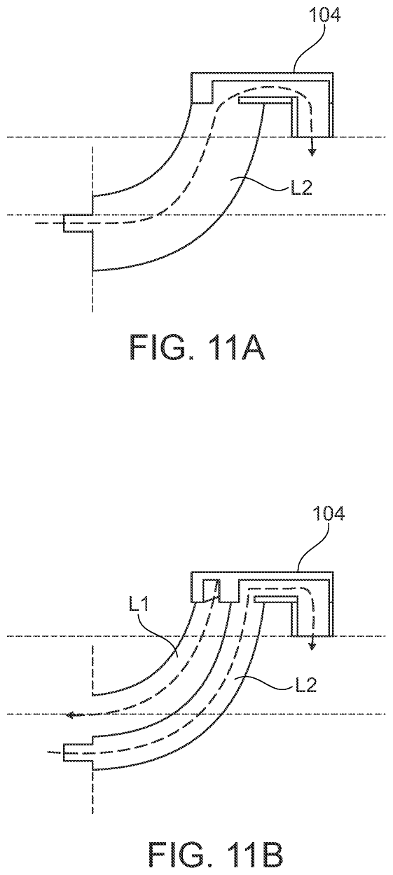

FIGS. 11A-B show longitudinal sectional views through connectors of the bag according to FIGS. 10A-B and

FIG. 12 is a perspective view of a bag according to the prior art, and

FIG. 13 is a perspective view of a bag with the connector removed according to the prior art.

The left-hand view in FIG. 1 (FIG. 1A) is a cross-sectional view of the connector K.

The connector K comprises the connection element 104, which in turn comprises a first projection F1 and a second projection F2, which extend from different sides of the central portion M of the connection element 104. The retaining elements 100, 102 are positioned on the projections F1 and F2 of the connection element 104 in the end portions thereof.

The central portion M constitutes the central part of the connection element 104, which can be the same distance from the two end regions of the projections F1 and F2 and from the retaining elements 100, 102.

Lines L1 and L2, which in turn are each connected to the retaining elements 100, 102, extend in the projections F1 and F2 starting from the central portion M. The lines L1 and L2 may be formed by the projections F1 and F2. Each of the retaining elements 100, 102 comprises a lumen 112, 114.

Each of the projections comprises a first edge or upper edge O and a second edge or lower edge U. The lower edge U is arranged at the bottom in the view in FIGS. 1A and 1s therefore referred to as the lower edge. Since a plan view of the connector K is shown in FIG. 1A, the first and the second edge or the upper edge and the lower edge are therefore edges of the upper face of the connector K shown in FIG. 1A. The first and the second edge or the upper edge O and the lower edge U transition into the central portion M without widening, as is clear from FIG. 1A, i.e. the central portion M is not thickened compared with the projections F1, F2 adjacent thereto.

Reference sign 106 shows the housing element of the connector K, which is fluidically connected to a bag opening (not shown) on one side and to the lines L1, L2 on the other side.

The right-hand view in FIG. 1 (FIG. 1B) is a perspective view of the connector K. It is clear from this view that the housing element 106 is narrower in its upper portion than in the regions adjacent thereto at the bottom, in which the housing element 106 has a constant width. Reference sign 106′ denotes the end region of the housing element 106, in which this is fluidically connected to the container or bag (not shown).

As shown schematically in FIG. 1A-B, each of the projections can transition into the central portion M without widening, i.e. the central portion M is not thickened compared with the projections F1, F2 adjacent thereto.

Identical or functionally identical elements have the same reference signs in the drawings.

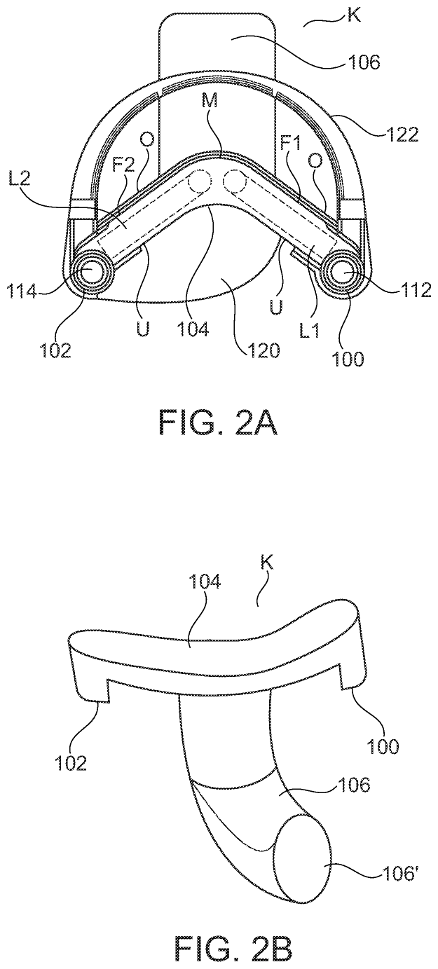

FIG. 2A-B show an embodiment in which the housing element 106 has substantially the same width in all regions. The opening 106′ to the bag is smaller than in the embodiment in FIG. 1A-B.

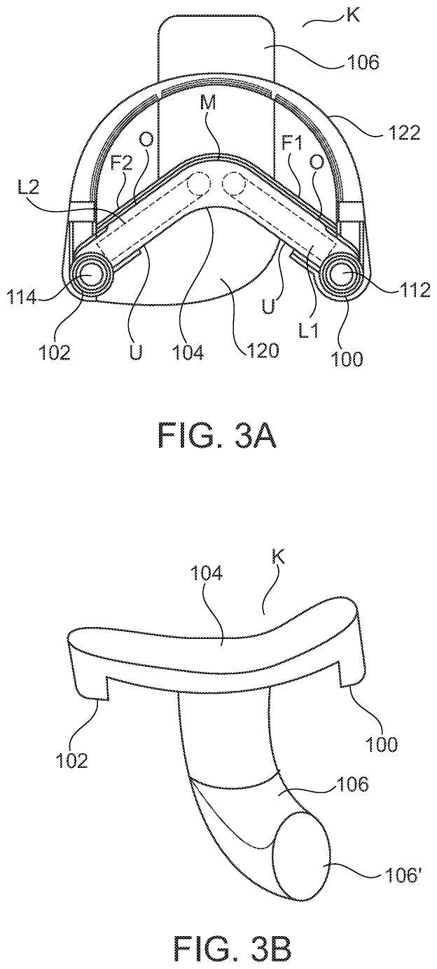

An embodiment is clear from FIG. 3A-B in which the housing element 106 has a smaller width in the upper regions than in the following regions, in which there is a gradual transition of the width in the vertical and/or horizontal portion of the housing element 106.

In the embodiments in FIG. 1-3, the connection element 104 is V-shaped, with the central region M forming the tip of the V shape and the projections F1, F2 forming the legs thereof.

FIGS. 4A-B show an embodiment in which the connection element 104 is designed to be linear. In this case, the central region M and the projections F1 and F2 lie on one line.

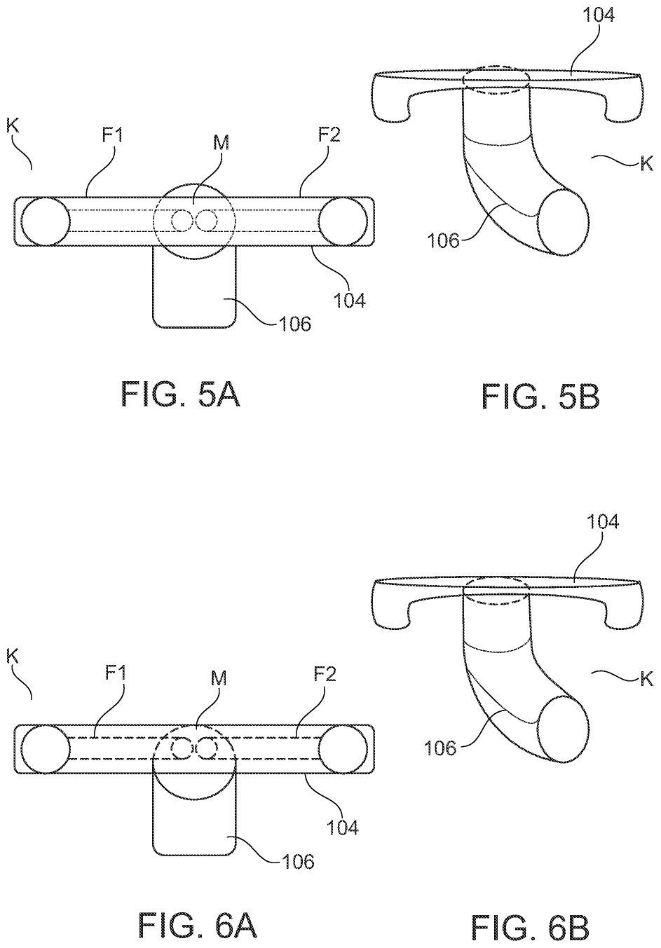

FIGS. 5A-B show an embodiment corresponding to FIG. 4, in which the upper region of the housing element adjacent to the connection element 104 has a larger width than the connection element 104. This is not the case according to FIG. 4. Here, the width of the upper region adjacent to the connection element 104 corresponds to that of the connection element 104.

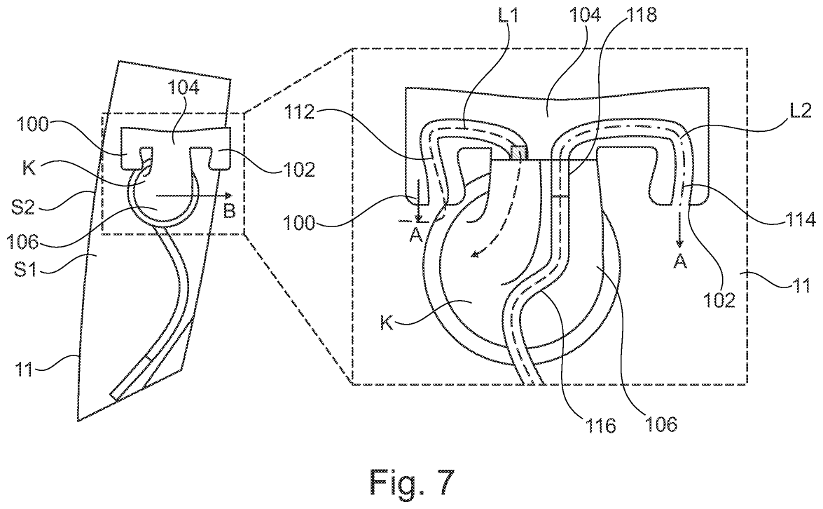

FIGS. 6A-B show an embodiment in which the upper region of the housing element 106 is not arranged centrally under the central region M of the connection element 104, but is offset therefrom. This results in the advantage that less space is required on one side of the connector K. This configuration can of course be combined with other embodiments of the invention and is therefore part of the invention.

FIG. 7 shows a bag 11 comprising two opposite side surfaces S1, S2, which e.g. consist of films. The bag opening in which the connector K is positioned is positioned in one of the side surfaces S1.

In principle, the connector may form a one-piece component of the bag 11. The invention also covers the fact that the connector K and the bag 11 form two parts which are or can be interconnected so as to be fluid-tight in a suitable manner, e.g. by screwing, latching, etc.

The connector K comprises two retaining elements 100, 102 having a first orientation A which are interconnected by a central connection element 104. The housing element 106 is arranged at the bottom of this central connection element 104, has a curvature and opens into the bag opening, with the bag opening having a surface normal B which is not parallel to the first orientation A. Preferably, the orientations A and B are perpendicular to one another.

A bag inlet is positioned in the first retaining element 100 in the form of the lumen 112. This first lumen serves to fluidically connect the bag to the outside, i.e. when it is connected to a dialysis machine.

The container outlet is positioned in the second retaining element 102 in the form of the lumen 114 for fluidically connecting the bag to the outside, i.e. when it is connected to a dialysis machine.

The first lumen 112 transitions into the first line L1 of the connection element 104, and the second line L2 of the connection element 104 transitions into the second line L2. The lines L1 and/or L2 may open or transition into lines of the housing element or may freely discharge into the interior of the bag.

The connection element 104 and the housing element 106 are preferably one-piece components of the connector K.

As is also clear from FIG. 7, the housing element 106 is guided downwards starting from the connection element 104, i.e. towards the bag opening.

The flow path of a fluid (gas and/or liquid) into the interior of the bag therefore takes place from the (not shown) dialysis machine into the first lumen 112 of the first retaining element 100, from there into the first line L1 of the connection element 104, and from there into the interior of the bag. A liquid, such as RO water, is preferably introduced into the bag in order to dissolve or dilute a concentrate contained in the bag. Alternatively or additionally, the introduction of a gas is conceivable and covered by the invention. This may be useful for producing a certain level of turbulence in the interior of the bag, which can accelerate the dissolution process of a solid concentrate.

The flow path of a fluid (gas and/or liquid) out of the interior of the bag therefore takes place through the tube 116 into the pipe piece 118, from there into the second line L2 of the connection element 104, and from there into the second lumen 114 of the second retaining element 102. From there, the fluid flows into the dialysis machine (not shown). This fluid is preferably a liquid dialysis concentrate, from which the ready-to-use dialysis solution is produced using one or more other concentrates and/or water. It is also conceivable for the fluid discharged from the bag to already be the ready-to-use dialysis solution.

The tubular portion 118 may also be designed to be longer and in particular may also be designed to be curved, such that it can be formed together with the curved housing in a demolding process.

Owing to the curved housing 106 that is guided downwards and the liquid lines L1, L2 guided therein, the bag inlet is offset downwards relative to the supports and the projecting length D of the bag in the upward direction is reduced.

FIG. 8A shows the situation in which a straight housing element does not extend downwards from the connection element. Therefore, there is a projecting length C between the lower edge of the retaining elements and the upper edge of the bag.

FIG. 8B shows a bag in which the retaining elements are offset upwards by means of the housing element 106, such that a shorter distance D between the lower edge of the retaining elements and the upper edge of the bag results than in FIG. 8A. A further reduction in this distance can be seen from FIG. 8C, in which the housing element 106 is longer than in FIG. 8B, such that the offset D′ is further reduced.

The curved housing element not only serves to reduce the distance between the lower edge of the retaining elements and the upper edge of the bag, but also to fluidically connect the interior of the bag to the line system of the dialysis machine when it is connected thereto.

The curved housing element 106 may have a quarter-circle shape starting from the opening of the bag. This makes it possible to produce this element using injection molding by means of an accordingly shaped core, since it can be removed by being turned about the circle axis.

As is clear from FIG. 1, a surface 120 can be provided in order to push a pin on the dialysis machine.

A grip 122 may be provided which in particular extends from one retaining element 100 to the other retaining element 102.

The upper face of the connector K may be closed by a welded-on film, but it may also be provided with a cover, which is connected to the main body by latching, bonding or squeezing.

The welded-on film and/or the cover may be the boundary (in the form of a wall) at the top for the lumen within the connector.

FIG. 9A-B show different configurations of the connector as well as cross-sectional views through the inlets and outlets into the interior of the bag (“in”) and out of the interior of the bag (“out”).

FIG. 9A shows an arrangement with a partition T between the region in which fluid flows into the bag and the region in which fluid flows out of the bag.

FIG. 9B shows a configuration in which a tube piece S is positioned upstream of the line L2 in the flow direction, the end of which is provided with a pipe piece.

FIG. 9C shows a configuration in which the connection element and thus also the lines L1 and L2 are designed to extend comparatively low down, meaning that good separation between fluid flowing in and fluid flowing out can be achieved with a sufficient distance. In addition, a pipe piece R is positioned upstream of the line L2.

According to FIG. 9D, the line L1 is arranged downstream of a tube piece S extending in the interior of the bag and the line L2 is arranged around the pipe piece R, which likewise extends into the interior of the bag in a projecting manner.

FIG. 9E shows an embodiment that is comparable with FIG. 9A, but in which the lines L1, L2 intersect in the connection element 104. Reference sign T again denotes the partition.

As is clear from FIGS. 10A and 10B, the connection element 104 may be angled, such that the bag hangs at a certain distance in front of the dialysis machine. By angling the connection element 104, the curved piece 106 can be designed to be shorter, and in particular a circular housing design can be achieved as a result.

As is clear from FIGS. 10A and 10B as well as FIGS. 11A and 11B, the end regions of the lines L1, L2 in the central connection element may be arranged one behind the other (FIGS. 10B and 11B) or beside one another (FIG. 10A and FIG. 11B).

1. A container for receiving a dry concentrate for producing a dialysis solution, comprising a container opening and a connector arranged on the container opening, wherein the container comprises a side wall and the container opening is arranged in this side wall, and wherein the connector comprises fixing means for fixing the container to a dialysis machine, which comprises a first retaining element having a container inlet and a second retaining element having a container outlet, wherein a connection element is provided on which the first and the second retaining element are arranged and which is provided with lines that are fluidically connected to the container inlet and outlet, and a housing element of the connector is provided which is fluidically connected both to said lines and to the container opening and extends from the connection element to the container opening, wherein the housing element comprises a curved or angled portion, wherein the connection element does not comprise a widened central portion in a plan view of the container.

2. The container according to claim 1, wherein the connection element comprises a first and a second projection, wherein the first projection is connected to the first retaining element and the second projection is connected to the second retaining element.

3. The container according to claim 2, wherein an upper edge of the first projection and an upper edge of the second projection transition into the central portion without widening.

4. The container according to claim 2, wherein a lower edge of the first projection and a lower edge of the second projection transition into the central portion without widening.

5. The container according to claim 2, wherein the central portion and the projections have the same width in a plan view of the container.

6. The container according to claim 1, wherein the connection element is designed to be angled or curved in a plan view of the container.

7. The container according to claim 2, wherein the projections and the central portion are arranged in a V shape.

8. The container according to claim 2, wherein the projections and the central portion are arranged on a line.

9. The container according to claim 1, wherein the connection element comprises a planar upper face which is arranged on the side opposite the side of the connection element on which the housing element is arranged.

10. The container according to claim 1, wherein the retaining elements are arranged so as to be offset upwards relative to the container opening.

11. The container according to claim 1, wherein the retaining elements have a first orientation, and in that the surface normal of the container opening has a second orientation, wherein said two above-mentioned orientations not extend in parallel, but rather at an angle to one another.

12. The container according to claim 1, wherein the housing part has a constant or increasing diameter and/or cross section starting from the connection element towards the container opening.

13. The container according to claim 1, wherein the housing element is circle-segment-shaped.

14. The container according to claim 1, wherein the upper face of the connector is closed by a film or a cover.

15. The container according to claim 1, wherein the connector comprises a separating means which is arranged to separate the liquid that enters the vessel through the container inlet from the liquid discharged from the vessel through the container outlet.

16. The container according to claim 1, wherein the ends of the lines of the connection element are arranged one behind the other or beside one another in a front view of the container.

17. A dialysis machine comprising a first connection for supplying the container of claim 1 with liquid and comprising a second connection for discharging liquid from the vessel, wherein the first and the second retaining element of the container are arranged at the first and the second connection, respectively.
